SEQUENCE LISTING (1 sequence)

SEQ ID NO: 1 - protein, 39 aa, Artificial Sequence
  Synthetic Construct
  MISC_FEATURE (2)..(2): Xaa at position 2 is nonnaturally occurring amino acid 2-Aminoisobutyric Acid
  MISC_FEATURE (13)..(13): Xaa at position 13 is nonnaturally occurring amino acid 2-Aminoisobutyric Acid
  MOD_RES (20)..(20): Lys at position 20 is chemically modified through conjugation to the epsilon-amino group of the K side-chain with (2-[2-(2-Amino-ethoxy)-ethoxy]-acetyl)2-(gamma- Glu)1-CO-(CH2)18-C O2H
  MOD_RES (39)..(39): Ser at position 39 is amidated as a C-terminal primary amide
YXEGTFTSDYSIXLDKIAQKAFVQWLIAGGPSSGAPPPS

(19) United States
(12) Patent Application Publication
(10) Pub. No.: US 2024/0277811 A1
(43) Pub. Date: Aug. 22, 2024

(54) METHODS FOR TREATING OBSTRUCTIVE SLEEP APNEA

(71) Applicant: Eli Lilly and Company, Indianapolis, IN (US)

(72) Inventor: Mathijs Christiaan Michael BUNCK, Indianapolis, IN (US)

(21) Appl. No.: 18/571,397

(22) PCT Filed: Jun. 21, 2022
(86) PCT No.: PCT/US2022/034212

§ 371 Date: Dec. 18, 2023

Related U.S. Application Data
(60) Provisional application No. 63/214,975, filed on Jun. 25, 2021.

(51) Int. Cl. A61K 38/22

(52) U.S. Cl. CPC A61K 38/22

(57) ABSTRACT

The present invention relates to methods for treating, preventing or delaying disorders relating to obstructive sleep apnea. The present invention relates to methods for treating, preventing or delaying obstructive sleep apnea. Provides a method of treatment for obstructive sleep apnea.

The present invention relates to the field of medicine. Provided are methods relating to treating, preventing, or delaying obstructive sleep apnea. Provided are methods relating to treating, preventing, or delaying a disorder associated with obstructive sleep apnea. Methods relating to treatment of obstructive sleep apnea.

Obstructive sleep apnea (OSA) is a breathing disorder associated with significant comorbidity and mortality. Obstructive sleep apnea is present in approximately 29% of adult Americans. Obstructive sleep apnea is even more prevalent in people living with obesity, i.e. a body mass index greater than 30 kg/m². Recurrent breathing interruptions in obstructive sleep apnea often impair the patient's ability to reach the deep, restorative phases of sleep. With obstructive sleep apnea, the muscles in the back of the throat often relax, allowing the soft palate to collapse and form a narrowed airway. Patients may snort, choke or gasp in attempts to open the airway. Patients with obstructive sleep apnea have an increased risk of cardiovascular morbidity and mortality.

Currently available therapeutic approaches have shown moderate success in treating the clinical signs and symptoms of OSA (that is, snoring and excessive daytime sleepiness) but have failed to address the underlying pathophysiology of the disease and, more importantly, the cardiovascular (CV) morbidity and mortality associated with OSA. Typical treatment for obstructive sleep apnea is continuous positive airway pressure (CPAP) using an air pump and face mask to apply mild air pressure to help maintain an open airway during sleep. Positive airway pressure (PAP) is the generally accepted treatment for obstructive sleep apnea; however, recent clinical trials suggest that PAP may have no meaningful effect on myocardial infarction, stroke, and mortality.

Many patients stop using their CPAP machines due to mask discomfort, dry nose, red eyes, and nasal congestion. Further the vibrating noise from the machine may be a nuisance for the sleep of the patient and a bed mate.

There is currently no Food and Drug Administration guidance for industry for the treatment of sleep apnea. Pharmaceutical treatments currently approved are indicated for the treatment of excessive sleepiness in narcolepsy and obstructive sleep apnea; however, none are currently approved for the treatment of the underlying pathophysiology of obstructive sleep apnea.

There is a need for a pharmacological treatment for sleep apnea.

The present invention provides methods for treating, preventing or delaying obstructive sleep apnea.

Accordingly, the present invention provides a method of treating, preventing or delaying development of sleep apnea, comprising administering tirzepatide in a therapeutically effective amount to the patient. In an embodiment, tirzepatide is administered once weekly to the patient in need of treatment for obstructive sleep apnea. In an embodiment, tirzepatide is administered to the patient once weekly for at least 4 weeks. In an embodiment, tirzepatide is administered to the patient once weekly for at least 12 weeks. In an embodiment, tirzepatide is administered to the patient once weekly for at least 20 weeks. In an embodiment, tirzepatide is administered to the patient once weekly for at least 52 weeks. In an embodiment, tirzepatide is administered to the patient for 2 years.

In another aspect, the present invention provides a method of preventing or delaying obstructive sleep apnea in a patient, comprising administering tirzepatide in a therapeutically effective amount to the patient. In an embodiment, tirzepatide is administered once weekly to the patient in need of treatment for obstructive sleep apnea. In an embodiment, tirzepatide is administered to the obstructive sleep apnea patient once weekly for at least 4 weeks. In an embodiment, tirzepatide is administered to the obstructive sleep apnea patient once weekly for at least 12 weeks. In an embodiment, tirzepatide is administered to the obstructive sleep apnea patient once weekly for at least 20 weeks. In an embodiment, tirzepatide is administered to the obstructive sleep apnea patient once weekly for at least 52 weeks. In an embodiment, tirzepatide is administered to the obstructive sleep apnea patient for 2 years.

In another aspect, the present invention provides a method of improving glycemic control and treating, preventing or delaying obstructive sleep apnea in a patient in a patient with and/or without type 2 diabetes mellitus, comprising administering tirzepatide in a therapeutically effective amount to the patient once weekly.

In another aspect, the present invention provides a method of improving glycemic control in a patient with type 2 diabetes mellitus and at risk for obstructive sleep apnea, comprising administering tirzepatide in a therapeutically effective amount to the patient once weekly, wherein the method provides a reduction in the risk of the patient experiencing sleep apnea.

In another aspect, the present invention provides tirzepatide for use in treating, preventing or delaying development of a obstructive sleep apnea in a patient, comprising administering tirzepatide in a therapeutically effective amount to the patient once weekly.

In another aspect, the present invention provides use of tirzepatide for the preparation of a medicament for treating, preventing or delaying development of a cognitive disorder in a patient, comprising administering tirzepatide in a therapeutically effective amount to the patient once weekly.

The present invention provides methods for treating, preventing or obstructive sleep apnea, comprising administering an effective amount of tirzepatide, or a pharmaceutically acceptable salt thereof to a patient in need of such treatment.

Accordingly, the present invention provides a method of treating, preventing or delaying development of obstructive sleep apnea in a patient, comprising administering an effective amount of tirzepatide, or a pharmaceutically acceptable salt thereof, to the patient once weekly.

In another aspect, the present invention provides a method of improving glycemic control and treating, preventing or delaying obstructive sleep apnea in a patient in a patient with type 2 diabetes mellitus, comprising administering an effective amount of tirzepatide, or a pharmaceutically acceptable salt thereof, to the patient once weekly.

In another aspect, the present invention provides a method of improving glycemic control and treating, preventing or delaying obstructive sleep apnea in a patient in a patient with type 2 diabetes mellitus, comprising administering an effective amount of tirzepatide, or a pharmaceutically acceptable salt thereof, to the patient once weekly.

In another aspect, the present invention provides a method of improving glycemic control in a patient with obese weight and at risk for obstructive sleep apnea, comprising administering an effective amount of tirzepatide, or a pharmaceutically acceptable salt thereof, to the patient once weekly, wherein the method provides a reduction in the risk of the patient experiencing sleep apnea.

In another aspect, the present invention provides a method of improving glycemic control in a patient with obese weight and at risk for obstructive sleep apnea, comprising administering an effective amount of tirzepatide, or a pharmaceutically acceptable salt thereof, to the patient once weekly, wherein the method provides a reduction in the risk of the patient experiencing obstructive sleep apnea.

In another aspect, the present invention provides a method of improving weight management in a patient with obesity and at risk for obstructive sleep apnea, comprising administering an effective amount of tirzepatide, or a pharmaceutically acceptable salt thereof, to the patient once weekly, wherein the method provides a reduction in the risk of the patient experiencing obstructive sleep apnea.

In another aspect, the present invention provides a method for treating obstructive sleep apnea in patient at risk for obstructive sleep apnea, comprising administering an effective amount of tirzepatide, or a pharmaceutically acceptable salt thereof, to the patient once weekly, wherein the patient's weight is within a normal weight range for the patient.

In another aspect, the present invention provides a method of improving weight management in a patient with obesity and at risk for obstructive sleep apnea, comprising administering an effective amount of tirzepatide, or a pharmaceutically acceptable salt thereof, to the patient once weekly, wherein the method provides a reduction in the risk of the patient experiencing obstructive sleep apnea.

In another aspect, the present invention provides tirzepatide, or a pharmaceutically acceptable salt thereof, for use in treating, preventing or delaying development of obstructive sleep apnea in a patient, comprising administering an effective amount of tirzepatide to the patient once weekly.

In another aspect, the present invention provides use of tirzepatide for the preparation of a medicament for treating, preventing or delaying development of obstructive sleep apnea in a patient, comprising administering tirzepatide, or a pharmaceutically acceptable salt thereof, in a therapeutically effective amount to the patient once weekly.

U.S. Pat. No. 9,474,780 describes and claims tirzepatide. When used herein, the term “tirzepatide” refers to any GIP/GLP-1 receptor agonist having the amino acid sequence of SEQ ID NO:1, including any protein that is the subject of a regulatory submission seeking approval of a GIP/GLP-1 receptor agonist product which relies in whole or part upon data submitted to a regulatory agency by Eli Lilly and Company relating to tirzepatide, regardless of whether the party seeking approval of said protein actually identifies the protein as tirzepatide or uses some other term. Tirzepatide agonizes the GIP/GLP-1 receptors resulting in stimulation of insulin synthesis and secretion, and has been shown to provide improved glycemic control in T2DM patients.

The methods provided herein may be most effective in patients at relatively higher risk for experiencing obstructive sleep apnea. In certain embodiments, such patients are those having one or more of: T2DM; hypertension; elevated cholesterol and/or obesity.

In certain embodiments, such patients have established cardiovascular disease; and/or one or more risk factors for major adverse cardiovascular events. When used herein, the term “major adverse cardiovascular events” refers to cardiovascular death, non-fatal myocardial infarction and non-fatal stroke. These events are also sometimes referred to as MACE or MACE-3 events. The first to occur of any of these events is a composite endpoint frequently used in cardiovascular outcomes trials.

When used herein in relation to major adverse cardiovascular events, the term “risk factors” refers to characteristics of patients understood to increase their risk for a major adverse cardiovascular event. Such risk factors include in particular any of the following: current tobacco use (any form of tobacco); use of at least 1 approved lipid modifying therapy (e.g., statins such as atorvastatin, rosuvastatin, simvastatin, pravastatin, lovastatin, fluvastatin or pitavastatin; PCSK9 inhibitors, such as evolocumab or alirocumab; and ezetimibe) to treat hypercholesterolemia or a documented untreated low-density lipoprotein cholesterol (LDL-C)≥3.4 mmol/L (130 mg/dL) within the past 6 months; documented treated or untreated high-density lipoprotein cholesterol (HDL-C)<1.0 mmol/L (40 mg/dL) for men and <1.3 mmol/L (50 mg/dL) for women or triglycerides >2.3 mmol/L (150 mg/dL) within the past 6 months; use of at least 1 blood pressure medication to treat hypertension (e.g., angiotensin converting enzyme (ACE) inhibitors, angiotensin receptor blockers (ARBs), thiazidelike diuretics, and dihydropyridine calcium channel blockers) or untreated systolic blood pressure (SBP) ≥140 mm Hg or diastolic blood pressure (DBP) ≥95 mmHg; measured waist-to-hip ratio >1.0 for men and >0.8 for women.

As used herein “improved weight management” means that the patient weight is within or closer to the clinically defined normal weight range for the patient. “Normal weight” for a particular patient may be determined by a clinician considering applicable considerations that are well known to the skilled clinician. Typically, improved weight management means that the patient loses weight to reach a weight that is within, or closer to, the desired weight range for the patient. As used herein “normal weight range” shall be a weight that a skilled clinician determines to be the normal weight for a particular patient. The normal weight range may vary based on the height of the patient and other factors considered by the skilled clinician in weight assessment. As used herein “Obese weight” means that the patient has a BMI greater than or equal to 30 kg/m².

When used herein, the terms “treatment,” “treat,” “treating,” and the like, are meant to include slowing or attenuating the progression of a disease, condition or disorder. These terms also include alleviating, ameliorating, attenuating, eliminating, or reducing one or more symptoms of a disorder or condition, even if the disorder or condition is not actually eliminated and even if progression of the disorder or condition is not itself slowed or reversed. When used herein, the terms “prevent,” “preventing,” “prevention,” and the like, are meant to include avoidance of the onset of a disease, condition, disorder or symptom. When used herein, the terms “delay,” “delaying,” and the like, are meant to include increasing the duration of time that occurs until onset of a disease, condition, disorder or symptom.

When used herein in connection with multiple outcomes, the term “composite” refers to the first to occur of any of the outcomes.

When used herein, the term “hazard ratio” refers to a measure of the relative rate of progression to an endpoint as compared to a control group. In outcome-based clinical trials, a reduction in the hazard ratio for a test arm as compared to the control indicates the therapy used in the test arm reduces the risk of the endpoint, in the case of the studies described herein, major adverse cardiovascular events.

“Therapeutically effective amount” means the amount of tirzepatide for the methods and uses of the present invention or pharmaceutical composition comprising tirzepatide for the methods and uses of the present invention that will elicit the biological or medical response of or desired therapeutic effect on the patient that is being sought by the researcher, medical doctor, or other clinician. An effective amount of tirzepatide may vary according to factors such as the disease state, age, sex, and weight of the individual, and the ability of tirzepatide to elicit a desired response in the individual. An effective amount is also one in which any toxic or detrimental effect is outweighed by the therapeutically beneficial effects. In certain embodiments, the therapeutically effective amount of tirzepatide for use in the methods described herein is selected from the group consisting of 5, 10 and 15 mg. In certain embodiments, the therapeutically effective amount of tirzepatide is 5.0 mg. In certain embodiments, the therapeutically effective amount of tirzepatide is 10.0 mg. In preferred embodiments, the therapeutically effective amount of tirzepatide is 15.0 mg. In an embodiment tirzepatide is administered as a pharmaceutically acceptable salt.

Additional embodiments of the present invention are described below:

In an embodiment is, a method of treating, preventing or delaying development of obstructive sleep apnea in a patient, comprising administering tirzepatide, or a pharmaceutically acceptable salt thereof, in a therapeutically effective amount to the patient once weekly. In an embodiment, the sleep apnea is obstructive sleep apnea.

In an embodiment is, a method of preventing or delaying obstructive sleep apnea in a patient, comprising administering tirzepatide, or a pharmaceutically acceptable salt thereof, in a therapeutically effective amount to the patient once weekly.

In an embodiment, is a method of improving glycemic control and treating, preventing or delaying obstructive sleep apnea in a patient with type 2 diabetes mellitus, comprising administering tirzepatide, or a pharmaceutically acceptable salt thereof, in a therapeutically effective amount to the patient once weekly.

In an embodiment, the method results in a reduction in the risk of the patient experiencing obstructive sleep apnea. In an embodiment the method results in a reduction in the risk of the patient experiencing obstructive sleep apnea.

In an embodiment is a method of improving glycemic control in a patient with type 2 diabetes mellitus, comprising administering tirzepatide, or a pharmaceutically acceptable salt thereof, in a therapeutically effective amount to the patient once weekly, wherein the method provides a reduction in the risk of the patient experiencing sleep apnea.

The method of any of the above embodiments wherein the patient has type 2 diabetes mellitus. The method of any of the above embodiments wherein the patient has obese weight.

The method of any of the above embodiments wherein the s obstructive sleep apnea patient has one or more of: T2DM and obese weight.

In an embodiment, the obstructive sleep apnea patient has either: obese weight with other metabolic disorders or obese weight with no other metabolic abnormalities.

In an embodiment, the obstructive sleep apnea patient has obese weight but without other metabolic abnormalities.

In an embodiment, the risk factors for cardiovascular disease are selected from the group consisting of: current tobacco use (any form of tobacco); use of at least 1 approved lipid modifying therapy to treat hypercholesterolemia or a documented untreated low-density lipoprotein cholesterol (LDL-C)≥3.4 mmol/L (130 mg/dL) within the past 6 months; documented treated or untreated high-density lipoprotein cholesterol (HDL-C)<1.0 mmol/L (40 mg/dL) for men and <1.3 mmol/L (50 mg/dL) for women or triglycerides >2.3 mmol/L (150 mg/dL) within the past 6 months; use of at least 1 blood pressure medication to treat hypertension or untreated systolic blood pressure (SBP) ≥140 mm Hg or diastolic blood pressure (DBP) ≥95 mmHg; measured waist-to-hip ratio >1.0 for men and >0.8 for women.

In an embodiment, the obstructive sleep apnea patient's Kansas City Cardiomyopathy Questionnaire Clinical Summary Score (KCCQ-CSS) improves with tirzepatide treatment. In an embodiment, the improved KCCQ-CSS correlates with a net clinical benefit.

In an embodiment, the risk of the occurrence of a composite of the following outcomes is reduced: hospitalization for obstructive sleep apnea or death.

In an embodiment, the risk of death or hospitalization for a condition related to sleep apnea is reduced in a patient treated with an effective amount of tirzepatide, or a pharmaceutically acceptable salt thereof.

In an embodiment, the risk of the occurrence of a composite of the following outcomes is reduced: type 2 diabetes and obstructive sleep apnea.

In an embodiment, the risk of the occurrence of a composite of the following outcomes is reduced: obstructive sleep apnea, myocardial infarction, or death.

In an embodiment, the risk of the occurrence of a composite of the following outcomes is reduced: obstructive sleep apnea, myocardial infarction, or death.

In an embodiment, the amount of tirzepatide is selected from the group consisting of about 5.0 mg, about 10.0 mg and about 15.0 mg.

In an embodiment, the amount of tirzepatide is about 5.0 mg.

In an embodiment, the amount of tirzepatide is about 10.0 mg.

In an embodiment, the amount of tirzepatide is about 15.0 mg.

In an embodiment, the patient is less than 50 years old In an embodiment, the patient is 30 years old or less.

In an embodiment, once weekly administration of tirzepatide is continued for at least 4 weeks. In an embodiment, once weekly administration of tirzepatide is continued for at least 12 weeks. In an embodiment, once weekly administration of tirzepatide is continued for at least 20 weeks. In an embodiment, once weekly administration of tirzepatide is continued for at least 52 weeks.

In an embodiment, once weekly administration of tirzepatide is continued for at least 2 years.

In an embodiment, once weekly administration of tirzepatide is continued for at least 3 years.

In an embodiment, once weekly administration of tirzepatide is continued for at least 4 years.

In an embodiment, once weekly administration of tirzepatide is continued for approximately 5 years.

In an embodiment, once weekly administration of tirzepatide is continued for at least 5.5 years.

In an embodiment, tirzepatide, or a pharmaceutically acceptable salt thereof, is administered using a dose increasing protocol.

In an embodiment, the patient is also administered the standard of care for reducing the risk of major adverse cardiovascular events.

In an embodiment, the patient is also administered the standard of care for treating the symptoms of conditions comorbid with sleep apnea.

In an embodiment, the patient is also administered a beta blocker.

In an embodiment, the patient is also administered a calcium channel blocker.

In an embodiment, the patient is also administered a diuretic.

In an embodiment, the patient is also administered an antithrombotic agent.

In an embodiment, the patient is also administered aspirin.

Tirzepatide, or a pharmaceutically acceptable salt thereof, for use in any of the above embodiments.

Use of tirzepatide, or a pharmaceutically acceptable salt thereof, in the preparation of a medicament for any of the above embodiments.

Further embodiments are described in the examples below, which are not to be construed as limiting.

### EXAMPLES

A randomized double-blind placebo-controlled trial of a maximum tolerated dose (MTD) of tirzepatide (10 or 15 mg) in patients with moderate-to-severe OSA, as diagnosed by home sleep study (HSS) or polysomnography (PGS), and obesity who are currently being treated with PAP therapy.

A randomized double-blind placebo-controlled trial of MTD tirzepatide (10 or 15 mg) in patients with moderate-to-severe OSA, as diagnosed by HSS or PGS, and obesity who are not currently being treated with PAP therapy.

The enrollment criteria, set forth in Table 1 below, are designed to include participants who are similar to patients seen within a sleep practice and/or at the primary case physicians office, who are obese and diagnosed with obstructive sleep apnea:

TABLE 1
Enrollment Criteria.
Key inclusion criteria

BMI >=30 kg/m²
Signed informed consent

The study is designed to consist of a screening visit followed by a treatment period. Patients are randomized to tirzepatide 5, 10, or 15 mg (dosed using an increasing dose protocol) or placebo and followed at approximately 4-week intervals. Study procedure includes an overnight research sleep study, i.e. polysomnography (PSG), to measure change in apnea-hypopnea index (AHI) from diagnostic sleep study completed prior to enrollment or, if needed, the baseline research sleep study.

TABLE 2
Study endpoints.

| | |
|---|---|
| Study 1: Patients not on PAP | Study 2: Patients on PAP |

| | |
|---|---|
| Primary | Superiority of tirzepatide (MTD) to placebo for the mean |
| Endpoint | % decrease in AHI from baseline |
| Key | Superiority of tirzepatide (MTD) to placebo for the |
| Secondary | following endpoints from baseline: |
| Endpoints | % of patients with ≥50% AHI reduction |
| | % of patients with AHI <5 or AHI 5-14 with Epworth |
| | Sleepiness Scale (ESS) ≤10 (objective measure for |
| | discontinuation of PAP therapy) |
| | % of patients with improved ESS (categorical shift) and |
| | Functional Outcomes of Sleep Questionnaire (FOSQ) |
| | (mean change) score |
| | Mean % change in body weight |

Analyses include assessment of the effects of tirzepatide on sleep apnea, as measured by AHI. Daytime sleepiness is assessed using a questionnaire.

Statistical analyses are completed using SAS software.

### Example 2

Change in apnea-hypopnea index (AHI) from baseline. This study will measure the change in the number of apneas plus hypopnea events per hour of sleep from baseline at the end of the study. The study will assess patients diagnosed with obstructive sleep apnea. The treatment groups will be compared to a placebo group that will continue to receive standard of care for their obstructive sleep apnea as prescribed by their managing physician. Each participant will have at least three study visits, with at least two overnight sleep studies. The study will include a periodic phone visit with a questionnaire to assess daytime sleepiness. The study will measure the change in apnea hypopnea index from baseline.

### Example 3

In this randomized, double-blind trial, normoglycemic participants with obese weight and moderate obstructive sleep apnea (AHI 15 to 29.9 events per hour) or severe obstructive sleep apnea (AHI more than 30 events per hour) and who are unwilling/unable to use CPAP therapy are randomized for 52 weeks to tirzepatide 5 mg, 10 mg, or 15 mg, using increase dose regimen, and placebo. Baseline characteristics are similar between groups.

### Example 4

A multicenter, randomized, parallel-arm, double-blind, placebo-controlled, Phase 3 study with 52-week treatment duration conducted under a basket-design, investigates the effects of treatment with weekly (QW) tirzepatide at the maximum tolerated dose (MTD) (10 mg or 15 mg), compared with placebo in participants who have moderate-to-severe OSA and obesity.

The clinical trial, Group 1 patient group, includes participants who are unwilling or unable to use PAP therapy.

The clinical trial, Group 2 patient group, includes participants who are on PAP therapy for at least 3 months at time of screening and plan to continue PAP therapy during the study.

Participants are assigned to the group reflecting their current PAP usage. The participants are then randomly assigned 1:1 to treatment or placebo.

Approximately 412 participants are randomly assigned to study intervention across the entire master protocol, with approximately 206 participants randomly assigned to study intervention in each investigational subgroup.

An upper limit of approximately 70% enrollment of male participants ensures a sufficiently large sample of female participants.

The study interventions are:
tirzepatide at the MTD (10 mg or 15 mg) SC QW, or
placebo.

The expected total duration of study participation for each participant, including screening and the post-treatment follow-up periods, is 60 weeks across the following study periods:
Screening: 4 weeks
Treatment: 52 weeks
Post-treatment follow-up: 4 weeks
   The maximum duration of treatment is 52 weeks.

The Functional Outcomes of Sleep Questionnaire (FOSQ) is used to assess effect of treatment. Subjects will be measured using FOSQ-10, FOSQ-30 (vigilance domain score), and the FOSQ-30 (activity level domain score). Further, each participant is assessed using the apnea hypopnea index, and assessed for remission or mild nonsymptomatic OSA.

Sequences

SEQ ID NO: 1
Tirzepatide
YX₁EGTFTSDYSIX₂LDKIAQKAFVQWLIAGGPSSGAPPPS

wherein X₁ is Aib; X₂ is Aib; K at position 20 is chemically modified through conjugation to the epsilon-amino group of the K side-chain with (2-[2-(2-Amino-ethoxy)-ethoxy]-acetyl)₂-(γGlu)₁-CO—(CH₂)₁₈—CO₂H; and the C-terminal amino acid is amidated as a C-terminal primary amide.

We claim:

1. A method of treating, preventing or delaying development of obstructive sleep apnea, comprising administering an effective amount of tirzepatide, or a pharmaceutically acceptable salt thereof, to a patient in need thereof.

2. The method as claimed by claim 1, wherein the sleep apnea is obstructive sleep apnea.

3. A method of preventing or delaying sleep apnea in a patient, comprising administering an effective amount of tirzepatide, or a pharmaceutically acceptable salt thereof, to the patient once weekly.

4. The method as claimed by claim 1 wherein the patient has type 2 diabetes mellitus.

5. The method as claimed by claim 1 wherein the patient is in need of improved weight management.

6. The method as claimed by claim 1 wherein the patient weight is obese.

7. A method of improving weight management and preventing or delaying obstructive sleep apnea in a patient with obese weight, comprising administering an effective amount of tirzepatide or a pharmaceutically acceptable salt thereof, to the patient once weekly.

8. The method as claimed by claim 1, wherein the method results in a reduction in the risk of the patient experiencing obstructive sleep apnea.

9. The method as claimed by claim 1 wherein the method results in a reduction in the risk of the patient experiencing obstructive sleep apnea.

10. The method as claimed by claim 1, wherein the patient's risk of the occurrence of a composite of the following outcomes is reduced: obstructive sleep apnea, transient ischemic attack or death.

11. The method as claimed by claim 1 wherein the therapeutically effective amount of tirzepatide is selected from the group consisting of about 5.0 mg, about 10.0 mg and about 15.0 mg.

12. The method as claimed by claim 11 wherein the therapeutically effective amount of tirzepatide is about 15.0 mg.

13. The method as claimed by claim 11 wherein the therapeutically effective amount of tirzepatide is about 10.0 mg.

14. The method as claimed by claim 11 wherein the therapeutically effective amount of tirzepatide is about 5.0 mg.

15. The method as claimed by claim 11, wherein once weekly administration of tirzepatide is continued for at least 2 years.

16. The method as claimed by claim 1 wherein the patient in need of treatment for sleep apnea has either: multiple cardiovascular risk factors without established cardiovascular disease; or established cardiovascular disease.

17. The method of claim 16 wherein the sleep apnea is obstructive sleep apnea.

18. The method as claimed by claim 17 wherein the patient's weight is obese.

19. The method as claimed by claim 1 wherein the patient's weight is not obese.

20. A method of improving weight management and preventing or delaying sleep apnea in a patient in a patient with type 2 diabetes mellitus, comprising administering an effective amount of tirzepatide or a pharmaceutically acceptable salt thereof, to the patient once weekly.

21. The method as claimed by claim 1, wherein the method provides a reduced risk of hospitalization for myocardial infarction.

22. The method as claimed by claim 1, wherein tirzepatide is administered as a pharmaceutically acceptable salt.

23. Tirzepatide, or a pharmaceutically acceptable salt thereof for use in the treatment of sleep apnea comprising administering tirzepatide in a therapeutically effective amount to the patient in need of such treatment.

24. Tirzepatide, or a pharmaceutically acceptable salt thereof as claimed by claim 23 wherein the sleep apnea is obstructive sleep apnea.
